(19) United States
(12) Patent Application Publication
(10) Pub. No.: US 2024/0318218 A1
(43) Pub. Date: Sep. 26, 2024

(54) SYSTEMS AND METHODS FOR ATP REGENERATION USING A SYNTHETIC ENZYME CASCADE AND NADH OXIDATION

(71) Applicant: The Trustees of Columbia University in The City of New York, New York, NY (US)

(72) Inventors: Scott BANTA, Fairfield, CT (US); Emma WILLETT, Coventry, CT (US)

(73) Assignee: The Trustees of Columbia University in The City of New York, New York, NY (US)

(21) Appl. No.: 18/610,436

(22) Filed: Mar. 20, 2024

Related U.S. Application Data
(60) Provisional application No. 63/567,224, filed on Mar. 19, 2024.
(60) Provisional application No. 63/453,238, filed on Mar. 20, 2023.

(51) Int. Cl. C12P 19/30

(52) U.S. Cl. CPC C12P 19/30

(57) ABSTRACT

Synthetic enzymatic cascades are provided that continuously produce adenosine triphosphate (ATP) from a variety of fuel sources. The cascades are prepared by expressing one or more NADH-dependent dehydrogenases, polyphosphate NAD+ kinases (PPNK), NADPH oxidases, and particular reversible ATP-NAD+ kinases (NADK). The NADH-dependent dehydrogenases oxidize fuel sources such as formate and methanol while converting NAD+ to NADH. The PPNKs convert the NADH to NADPH. The NADPH oxidases convert NADPH to NADP⁺. The NADKs then convert the NADP⁺ to NAD⁺ while also facilitating conversion of adenosine diphosphate (ADP) or adenosine monophosphate (AMP) to ATP. Human NADK exhibits high affinity for NAD⁺ and is thus impeded in the generation of ATP products via product inhibition. Thus, pigeon, duck, and cat NADK isoforms are implemented in the cascade instead. The cascades generate a low-cost, continuous source ATP product for use in numerous in vitro applications such as cell-free protein production.

### CROSS REFERENCE TO RELATED APPLICATION(S)

This application claims the benefit of U.S. Provisional Application Nos. 63/453,238, filed Mar. 20, 2023, and 63/567,224, filed Mar. 19, 2024, which are incorporated by reference as if disclosed herein in their entireties.

### STATEMENT ON FEDERALLY SPONSORED RESEARCH OR DEVELOPMENT

This invention was made with government support under 1844254 awarded by the National Science Foundation. The government has certain rights in the invention.

### BACKGROUND

Adenosine triphosphate (ATP) is a cofactor used in many biological processes including the synthesis of RNA and DNA, the activation of weakly reactive functional groups such as carboxylic acids and alcohols, and can be used as a post-translational modification signal in enzymes. ATP regeneration is an important bioengineering capability that can be used to support for cell-free protein synthesis (CFPS), pseudo-cell development, and other in vitro applications. Existing methods for regenerating ATP typically include expensive phosphate donors and ATP-synthase enzymes that use a proton gradient across a membrane.

Cell-free protein synthesis produces large-scale personalized medicine and virus-like particles and uses ATP as a substrate. A newly rising need for ATP is in the creation of pseudo cells. Designing a cell in vitro uses ATP as an energy source, e.g., for actuation and mobility. To regenerate ATP, eukaryotic cells use the oxidation of NADH during the citric acid cycle to produce a proton gradient in the intermembrane space of mitochondria. Some proposed methods for ATP regeneration mimic the TCA cycle and use a membrane, which increases complexity. Current ATP regeneration methods used in cell-free protein synthesis include enzymes such as creatine kinase and pyruvate kinase and use a constant supply of expensive phosphate donors (phosphocreatine, pyruvate kinase, phosphoenelpyruvate, etc.). A biomimetic artificial organelle combined with an ATP synthase and a proton pump has been constructed and intended for use in powering artificial cells. This method for ATP regeneration uses a membrane to create a proton gradient in the vesicle.

Artificial cells that synthesize ATP have been built, but a major limitation thus far is the cells' inability to survive for long periods of time. ATP alone appears insufficient to sustain an artificial cell. Only a few attempts to regenerate energy carriers, such as cofactors, in synthetic cells have been reported. Cell-free translation systems generally utilize high-energy phosphate compounds to regenerate the ATP necessary to drive protein synthesis. This limits widespread use and practical implementation of this technology in large quantities due to expensive reagent costs, accumulation of inhibitory byproducts, and pH change. Building up more complex reaction cascades can mean the use of more ATP, so several studies use ATP in their mixtures. A self-sustaining energy system can be a hallmark of a functionalized artificial cell.

The ATP-NAD⁺ kinase (NADK) is the only known enzyme capable of phosphorylating NAD(H) to NADP(H), and therefore it plays an important role in maintaining NAD(P)(H) homeostasis. All domains of life contain at least one NADK gene, and all of the commonly investigated isoforms have been measured, or assumed, to be irreversible.

The nicotinamide cofactors NAD(H) and NADP(H) are important biological redox mediators involved in a myriad of cellular processes including electron transfer in central metabolism. The 2′-phosphorylation is used by enzymes to discriminate between the cofactors such that anabolic and catabolic reactions can occur in the same space, without cross-reaction. NAD(H) also participates in a variety of non-redox reactions. For example, the sirtuins are a family of NAD⁺-dependent deacylases that regulate metabolism and are involved in cellular aging. The PARP family of proteins are also NAD⁺-dependent enzymes involved in DNA repair and preservation.

Referring now to FIG. 1, the biosynthesis of NAD⁺ can occur through i) a de novo pathway from dietary L-tryptophan (or L-aspartic acid in prokaryotes), ii) the Preiss-Handler pathway from dietary niacin, and iii) a salvage pathway that recovers NAD⁺. In the de novo pathway from amino acids, quinolinic acid is produced which also feeds into the Preiss-Handler pathway, leading to the production of NAD⁺. NADK can then phosphorylate NAD(H) to NADP(H). Knock-out experiments in bacteria have indicated that NADK function is important. Some NADKs are specific for the reduced or oxidized forms of NAD(H) and some NADKs can use polyphosphate or other nucleoside triphosphates as phosphate donors, while eukaryotic NADKs are more specific for ATP. Quantitative tracer analysis in a breast cancer cell line showed that NADK activity accounted for ˜10% of the NAD⁺ pool consumption flux.

Yeast NADK was first isolated in 1950 and homologs have subsequently been investigated from several other native sources. In 2001, the first NADK gene was cloned, and NADK genes from all domains have been cloned, expressed recombinantly, and characterized. NADK enzymes tend to be multimeric, and the human NADK is a homotetramer. Several NADK crystal structures are available including the human NADK (3PFN) as well as the enzymes from Mycobacterium tuberculosis, Listeria monocytogenes, Archaeoglobus fulgidus, and Thermotoga maritima.

The net reversible reaction for NADK is:

NAD⁺+MgATP²⁻≈NADP⁺+MgADP⁻

In the forward direction, a phosphate group is transferred from ATP to NAD⁺ to form ADP and NADP⁺. A divalent cation (often Mg²⁺) can be used for catalysis. A substrate-assisted catalysis mechanism has been proposed for the forward reaction of NADK, where ATP supplies the divalent cation which is chelated by the pyrophosphate of NAD⁺ and this complex enables the specific phosphorylation of the 2′-hydroxyl group, forming NADP⁺.

NADK activity has been hypothesized to play a large role in regulating NAD(P)(H) homeostasis in cells. Microbial NADKs can be subject to product inhibition and allosteric regulation by NAD(H) and/or NADP(H). For example, human NADK has a strong preference for NAD⁺ over NADH as a substrate, and is inhibited by NADH and NADPH but not NADP⁺. Plant NADKs can be regulated by calcium/calmodulin. Recently it was shown in mammalian cells that phosphorylation of the N-terminal domain of NADK by the protein kinase Akt regulates NADK activity and the NAD(H)/NADP(H) cellular ratio.

The NADK reaction is predicted to have an apparent equilibrium constant favoring the forward reaction (K′_{eq}=119), and in most cells the NAD(H) and ATP concentrations would be higher than the NADP(H) and ADP concentrations so reverse activity would generally be unlikely in healthy physiological states. However, without wishing to be bound by theory, there appears to be no thermodynamic reason why the enzymes should be irreversible, and no explanation has been provided as to why the human enzymatic activity is observed to be irreversible. Product inhibition experiments are sometimes performed to explore kinetic mechanisms in these enzymes, however reports of the characterization of the reverse activities of NADKs are uncommon.

### SUMMARY

Aspects of the present disclosure are directed to a method for continuously producing adenosine triphosphate (ATP). In some embodiments, the method includes expressing one or more NADH-dependent dehydrogenases, one or more polyphosphate NAD⁺ kinases (PPNK), one or more NADPH oxidases, and one or more ATP-NAD⁺ kinases (NADK); oxidizing one or more fuel sources while converting NAD⁺ to NADH via the NADH-dependent dehydrogenases; converting NADH to NADPH via the PPNKs; converting NADPH to NADP⁺ via the NADPH oxidases; and converting NADP⁺ to NAD⁺ while converting one of adenosine diphosphate (ADP) or adenosine monophosphate (AMP) to ATP via the NADKs. In some embodiments, converting NADH to NADPH via the PPNKs occurs in the presence of a monophosphate compound.

In some embodiments, the NADH-dependent dehydrogenase includes formate dehydrogenase, formaldehyde dehydrogenase, alcohol dehydrogenase, or combinations thereof. In some embodiments, the molar ratio of NADH-dependent dehydrogenase to fuel source is less than about 0.001:1. In some embodiments, the NADH-dependent dehydrogenase includes formate dehydrogenase, formaldehyde dehydrogenase, and alcohol dehydrogenase. In some embodiments, the NADH-dependent dehydrogenase includes formate dehydrogenase from C. boidinii. In some embodiments, the one or more PPNKs are from B. subtilis. In some embodiments, the NADPH oxidase is a water-forming NADPH oxidase (TPNOX) from L. brevis, includes glucose-6-phosphate dehydrogenase (G6PDH), or combinations thereof. In some embodiments, the NADK is pigeon NADK, duck NADK, cat NADK, or combinations thereof. In some embodiments, the fuel source includes formate, formaldehyde, methanol, glucose, glycerol, or combinations thereof. In some embodiments, the concentration of the fuel source is maintained above about 10 mM.

Aspects of the present disclosure are directed to a method of generating products via a synthetic enzymatic cascade. In some embodiments, the method includes preparing a reaction medium including a concentration of a fuel source, a first concentration of NAD⁺, and one or more NADH-dependent dehydrogenases; converting at least a portion of the NAD⁺ to a concentration of NADH; contacting one or more PPNKs with the concentration of NADH; converting at least a portion of the NADH to a concentration of NADPH; contacting one or more NADPH oxidases with the concentration of NADPH; converting at least a portion of the NADPH to a concentration of NADP⁺; contacting one or more NADKs with the concentration of NADP⁺ and a concentration of ADP; and converting at least a portion of the ADP to ATP. In some embodiments, the method includes converting at least a portion of the NADP⁺ to a second concentration of NAD⁺; and providing at least a portion of the second concentration of NAD+ to the reaction medium.

In some embodiments, the fuel source includes formate, formaldehyde, methanol, glucose, glycerol, or combinations thereof. In some embodiments, the molar ratio of NADH-dependent dehydrogenase to fuel source is less than about 0.001:1. In some embodiments, the NADK is pigeon NADK, duck NADK, cat NADK, or combinations thereof.

Aspects of the present disclosure are directed to a method for continuously producing ATP including expressing in a reaction medium a plurality of proteins including a formate dehydrogenase, a formaldehyde dehydrogenase, an alcohol dehydrogenase, pigcon NADK, a PPNK from B. subtilis, and TPNOX; administering one or more fuel sources, monohydrogen phosphate, and ADP to the reaction medium; converting the ADP to a concentration of ATP via the NADK; and providing at least a portion of the concentration of ATP to an in vitro process. In some embodiments, the medium further comprises an AMP phosphotransferase, and the method further comprises converting at least a portion of a concentration of AMP to ADP.

In some embodiments, the one or more fuel sources includes methanol. In some embodiments, the in vitro process includes cell-free protein production, biomimetic artificial organelles, artificial cells, smart dust, or combinations thereof. In some embodiments, the molar ratio of NADH-dependent dehydrogenase to fuel source is less than about 0.0001:1, and the concentration of the fuel source is maintained above about 10 mM.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic flowchart showing NAD⁺ biosynthesis pathways;

FIG. 2 is a chart of a method of continuously producing adenosine triphosphate (ATP) according to some embodiments of the present disclosure;

FIGS. 3A-3B are graphs showing results of rate kinetic experiments performed on ATP-NAD⁺ kinases (NADK) enzymes according to some embodiments of the present disclosure;

FIG. 4 is a graph showing the results of ATP production experiments utilizing synthetic enzymatic cascades according to some embodiments of the present disclosure;

FIGS. 5A-5B are graphs showing the results of ATP production experiments with polyphosphate and monophosphate reactants utilizing synthetic enzymatic cascades according to some embodiments of the present disclosure;

FIG. 6 is a graph showing the results of ATP production from adenosine diphosphate (ADP) utilizing synthetic enzymatic cascades according to some embodiments of the present disclosure;

FIG. 7 is a graph showing the results of synthetic enzymatic cascade sensitivity analyses performed on synthetic enzymatic cascades according to some embodiments of the present disclosure;

FIG. 8 is a graph showing the results of exemplary synthetic enzymatic cascades according to some embodiments of the present disclosure using methanol as a fuel;

FIG. 9 is a chart of a method of generating products via a synthetic enzymatic cascade according to some embodiments of the present disclosure;

FIG. 10 is a chart of a method of continuously producing ATP according to some embodiments of the present disclosure;

FIGS. 11A-11C portray the results of a cell free protein synthesis system (CFPS) used for the production of superfolder green fluorescent protein (GFP) and horseradish peroxidase (HRP) utilizing synthetic enzymatic cascades according to some embodiments of the present disclosure;

FIG. 12 is a graph showing the results of synthetic enzymatic cascade sensitivity analyses performed on synthetic enzymatic cascades according to some embodiments of the present disclosure;

FIG. 13 is a schematic flowchart showing ATP production by a synthetic enzymatic cascade according to some embodiments of the present disclosure; and

FIG. 14 is a schematic flowchart of a synthetic methanol oxidation cascade for use with a synthetic enzymatic cascade according to some embodiments of the present disclosure.

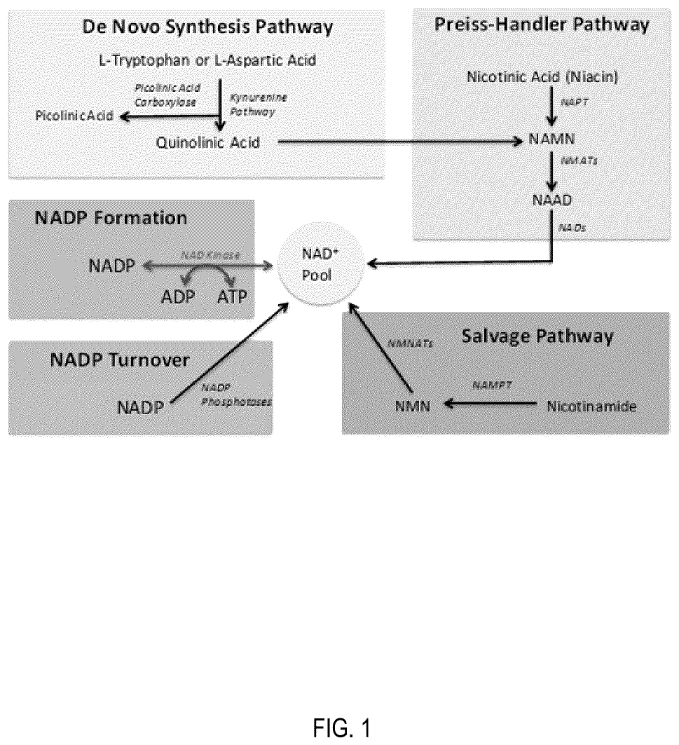

### DETAILED DESCRIPTION

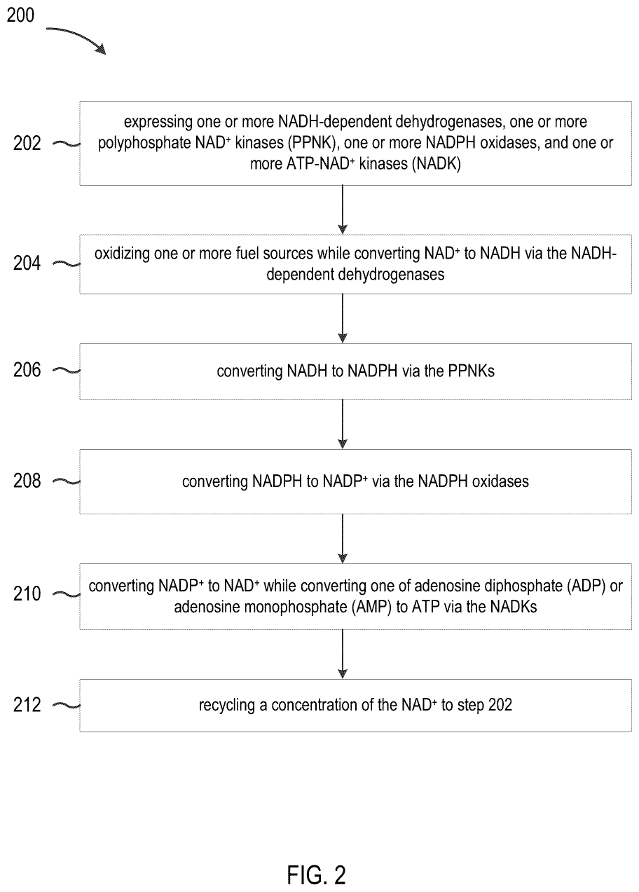

Referring again to FIG. 2, some embodiments of the present disclosure are directed to a synthetic enzymatic cascade that generates and/or regenerates adenosine triphosphate (ATP) from adenosine diphosphate (ADP), adenosine monophosphate (AMP), or combinations thereof. In some embodiments, the synthetic enzymatic cascade continuously generates/regenerates ATP, i.e., reactants are made available to the cascade in amounts sufficient such that an ATP product can be continuously or substantially continuously produced at a desired, predetermined rate.

Some embodiments of the present disclosure are directed to a method 200 for continuously producing ATP. At 202, a plurality of proteins are expressed. In some embodiments, the plurality of proteins include the proteins in the synthetic enzymatic cascade described above. In some embodiments, the plurality of proteins include one or more additional proteins in addition to those of the synthetic enzymatic cascade described above. In some embodiments, the plurality of proteins are recombinantly expressed in a target organism, e.g., a bacteria or eukaryote. In some embodiments, the plurality of proteins is expressed in a cell-free system. In some embodiments, the plurality of proteins includes one or more NADH-dependent dehydrogenases, one or more polyphosphate NAD+ kinases (PPNK), one or more NADPH oxidases, and one or more ATP-NAD+ kinases (NADK).

At 204, NADH-dependent dehydrogenases, e.g., at least a portion of those expressed at step 202, oxidize one or more fuel sources while converting NAD+ to NADH. In some embodiments, the fuel source includes formate, formaldehyde, methanol, glucose, glycerol, or combinations thereof. In some embodiments, the NADH-dependent dehydrogenases include formate dehydrogenase, formaldehyde dehydrogenase, alcohol dehydrogenase, or combinations thereof. In some embodiments, the NADH-dependent dehydrogenases include formate dehydrogenase, formaldehyde dehydrogenase, and alcohol dehydrogenase. In some embodiments, the NADH-dependent dehydrogenase includes formate dehydrogenase from C. boidinii. In some embodiments, the fuel sources are provided to the expression medium used in step 202, thus driving generation of a concentration of NADH in the expression medium. In some embodiments, the NADH-dependent dehydrogenases expressed at step 202 are removed from the expression medium and subsequently contacted with a concentration the fuels sources in a separate reaction medium.

In some embodiments, the molar ratio of NADH-dependent dehydrogenase to fuel source, e.g., in the expression medium, in a separate reaction medium, etc., is below about 0.0001:1. In some embodiments, the molar ratio of NADH-dependent dehydrogenase to fuel source is between about 0.0001:1 and about 0.000005:1. In some embodiments, the molar ratio of NADH-dependent dehydrogenase to fuel source is between about 0.0001:1 and about 0.00001:1. In some embodiments, the concentration of the fuel source, e.g., in the expression medium, reaction medium, etc., is maintained above about 0.1 mM. In some embodiments, the concentration of the fuel source is maintained above about 1 mM. In some embodiments, the concentration of the fuel source is maintained above about 10 mM.

At 206, the PPNKs, e.g., at least a portion of those expressed at step 202, convert a concentration of the NADH produced during step 204 to NADPH. In some embodiments, the one or more PPNKs are from B. subtilis. As discussed above, in some embodiments, the NADH is converted to NADPH in the expression medium. In some embodiments, the PPNKs expressed at step 202 are removed from the expression medium and subsequently contacted with a concentration of NADH in a separate reaction medium.

In some embodiments, step 206 occurs in the presence of one or more phosphate donors. In some embodiments, the phosphate donors include one or more polyphosphate compounds (also referred to herein as “polyphosphates”), one or more monophosphate compounds (also referred to herein as “monophosphates”), or combinations thereof. In some embodiments, the phosphate donors include sodium hexametaphosphate, monohydrogen phosphate, or combinations thereof. As discussed above, in some embodiments, the phosphate donors are present in the expression medium, in a separate reaction medium, or combinations thereof. In some embodiments, the concentration of the phosphate donor is maintained above about 1 mM. In some embodiments, the concentration of the phosphate donor is maintained above about 10 mM. In some embodiments, the concentration of the phosphate donor is maintained above about 50 mM.

At 208, the NADPH oxidases, e.g., at least a portion of those expressed at step 202, convert a concentration of the NADPH produced during step 206 to NADP⁺. The NADPH oxidases are highly specific for NADPH over NADH and this step provides a thermodynamic driving force for the cascade. In some embodiments, the conversion of NADPH to NADP⁺ produces water. In some embodiments, the NADPH oxidase includes a water-forming NADPH oxidase (TPNOX) from L. brevis, includes glucose-6-phosphate dehydrogenase (G6PDH), or combinations thereof. As discussed above, in some embodiments, the NADPH is converted to NADP⁺ in the expression medium. In some embodiments, the NADPH oxidases expressed at step 202 are removed from the expression medium and subsequently contacted with a concentration of NADPH in a separate reaction medium.

At 210, the NADKs, e.g., at least a portion of those expressed at step 202, convert a concentration of the NADP⁺ produced during step 208 to NAD⁺ while converting ADP and/or AMP to ATP, thus producing an ATP product. In some embodiments, the NADK is pigeon NADK, duck NADK, cat NADK, or combinations thereof. As discussed above, in some embodiments, the ADP/AMP is converted to the ATP product (and NADP⁺ converted to NAD⁺) in the expression medium. In some embodiments, the NADKs expressed at step 202 are removed from the expression medium and subsequently contacted with a concentration of ADP and/or AMP in a separate reaction medium.

Recombinantly-expressed NADKs were explored for their reversible activities. It was hypothesized that the reversibility of the pigeon enzyme would enable compensation for the high picolinic acid carboxylase (PC) activity present in pigeon livers, which inhibits NAD⁺ biosynthesis from dietary tryptophan. Duck and cat livers have higher PC activity than pigeon, and the recombinant duck and cat NADKs exhibit high activity in the reverse direction. It was determined that human NADK has an affinity for NAD⁺ that is about 600 times higher than the pigeon, duck and cat isoforms. Without wishing to be bound by theory, NAD⁺ serves as a product inhibitor for the reverse human NADK activity, which accounts for the observed irreversible behavior. While each of the NADKs explored were “reversible,” the reverse activity of the human enzyme is impeded via product inhibition. Thus, the use of pigeon NADK, duck NADK, and/or cat NADK in the synthetic enzymatic cascade, step 210, etc. advantageously enables the production of ATP that can be utilized in situ or removed as a product, while also continually generating an additional concentration of NAD⁺ and operating in those conditions.

In some embodiments, at 212, a concentration of the NAD⁺ generated at step 210 is recycled back to step 202 for conversion by the NADH-dependent dehydrogenase to a concentration of NADH, so that the synthetic enzymatic cascade can repeat and produce additional ATP product.

The synthetic enzymatic cascade is thermodynamically driven, in part, by fuel oxidation via dehydrogenases that regenerate NADH from NAD⁺. In exemplary embodiments, formate dehydrogenase (FDH) from C. boidinii is used for this step, alone or in combination with other dehydrogenases when utilizing, e.g., more energy dense fuel sources, such as formaldehyde dehydrogenase (formDH) and/or alcohol dehydrogenase (ADH). The dehydrogenases oxidize fuel sources including formate to CO₂ while producing NADH from NAD⁺. In an exemplary embodiment, a synthetic methanol oxidation cascade was shown to completely oxidize methanol which can be used to further regenerate NAD⁺ to NADH, as will be discussed in greater below.

In the exemplary embodiment, a PPNK from B. subtilis selectively converted the NADH formed to NADPH using a phosphate source, e.g., polyphosphate. The NADPH was then oxidized to NADP⁺ with TPNOX from L. brevis. A reversible NADK then converted ADP and NADP⁺ to ATP and NAD⁺. As discussed above, NADK enzymes are generally considered in the literature to irreversibly convert ATP. However, the pigeon and duck liver NADK enzymes are reversible (unlike human and rodent) and can be used in the cascade to regenerate ATP from ADP. The NAD⁺ produced was then recycled back to the dehydrogenases to complete the cascade.

The enzymes involved in the cascade were characterized. Kinetic parameters for the NADH-dependent dehydrogenases, PPNKs, NADPH oxidases, and NADKs included in the exemplary embodiments of the present disclosure are shown in Table I below.

TABLE I
Kinetic parameters for an exemplary 4-enzyme ATP regeneration cascade.

| Enzyme | k_{cat} (min⁻¹) | K_{M}^{a} (mM) | K_{M}^{b} (mM) | K_{I}^{a} (mM) | K_{I}^{b} (mM) |
|---|---|---|---|---|---|
| NADK* | 8.7 ± 3.6 | 0.73 ± 0.16 | 27 ± 10 | 0.27 ± 0.18 | 9.9 ± 2.5 |
| FDH** | (3.4 ± 0.1) × 10² | 0.030 ± 0.002 | 4.0 ± 0.06 | 0.22 ± 0.03 | — |
| PPNK*** | (5.6 ± 2.6) × 10² | 1.2 ± 0.6 | 0.15 ± 0.09 | 0.28 ± 0.19 | 0.034 ± 0.035 |
| TPNOX**** | (1.7 ± 0.1) × 10⁴ | 0.029 ± 0.001 | — | — | — |

*a isNADP and b is ADP; **a is NAD and b is formate; abbreviated sequential mechanism; ***a is NADH and b is poly(P); ****a is NADPH, Michaelis-Menten mechanism.

The ATP-producing NADK has been predominantly studied in the forward direction (consuming ATP) since the observed isoforms, such as the human NADK, are irreversible. Without wishing to be bound by theory, one form, pigeon NADK, follows a random bi-bi sequential mechanism and the kinetic parameters for the reverse direction are found in Table I.

Synthetic pigeon, duck, and cat NADK genes and a cloned human NADK gene all with C-terminal 6×-histidine tags were expressed E. coli and purified to apparent homogencity. The pigeon enzyme shares 85% sequence homology with the human enzyme, while the duck and pigeon enzymes are 96% sequence homology. The cat enzyme shares a 91% sequence homology to the human enzyme.

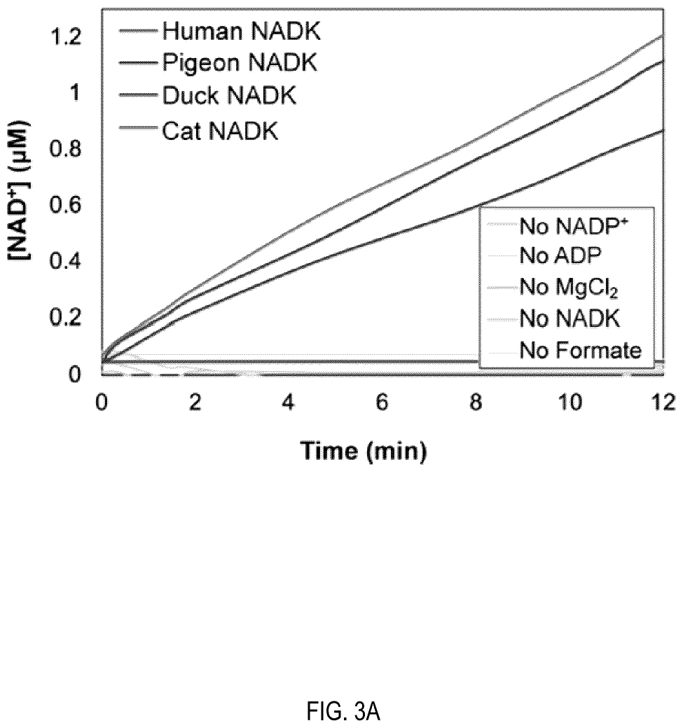

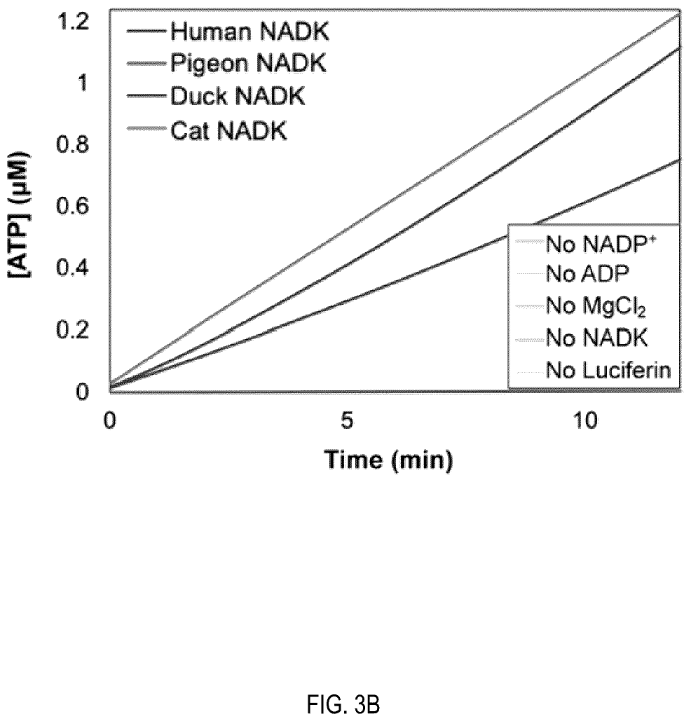

Referring now to FIGS. 3A-3B, initial rate kinetic experiments were performed with pigeon, duck, cat, and human NADK enzymes in both the forward and reverse directions. The pigeon, duck, and cat recombinant NADK enzymes exhibited reverse NADK activities. In FIG. 3B, the production of ATP was detected in a coupled reaction with firefly luciferase where ATP was converted to light, the relative luminescence (RLU) was monitored, and a standard curve was used to calculate the ATP concentration. Similarly, in FIG. 3A, NAD⁺ formation was measured in a coupled assay with FDH and the absorption of NADH was monitored. Removal of any of the substrates in both the forward and reverse direction showed no activity. Consistent with prior observations, the human NADK had no measurable activity in the reverse direction.

The FDH from C. boidinii was expressed recombinantly in a pEt28a plasmid and purified according to the materials and methods. The enzyme follows a sequential mechanism and the kinetic parameters are reported in Table I.

The PPNK from B. subtilis has been studied with both polyphosphate (polyphosphate glass with average chain length between 13-18) and ATP as substrates with NAD⁺. A recombinant PPNK protein was ligated with a 6×-histidine tag into a pET20b vector and expressed and purified. The reduced form, NADH, was also found to be active with PPNK with both ATP and poly(P) as substrates. Sodium metaphosphate was used as the polyphosphate source with NADH because it is cheap and has shown high activity with other PPNK isoforms. The initial rates were fit to a random bi-bi sequential mechanism and shown, e.g., in Table II. The kinetic parameters with NAD⁺ are also in Table II. Because the PPNK has activity with both ATP and NAD⁺, this enzyme was predicted to be the main source of cross-reactions. However, the PPNK has high activity with polyphosphate and NADH and is a good candidate for NADH phosphorylation (Tables I and II).

TABLE II
PPNK kinetic parameters for substrates NAD⁺, NADH, ATP, and poly(P).

| Substrates | k_{cat} (min⁻¹) | K_{M}^{NADH} (mM) | K_{M}^{b} (mM)** | K_{I}^{NADH} (mM) | K_{I}^{b} (mM)** |
|---|---|---|---|---|---|
| NADH, ATP | (2.6 ± 1.9) × 10² | 1.7 ± 1.4 | 1.40 ± 1.11 | 0.68 ± 0.12 | 0.56 ± 0.64 |
| NADH, poly(P)* | (5.6 ± 2.6) × 10² | 1.2 ± 0.6 | 0.15 ± 0.09 | 0.28 ± 0.19 | 0.034 ± 0.035 |

*polyphosphate is a sodium metaphosphate; **b is ATP and polyphosphate respectively.

In some embodiments, the final enzyme and the thermodynamic driving force of the cascade is the TPNOX from L. brevis. The kinetic parameters of this enzyme are reported in Table I.

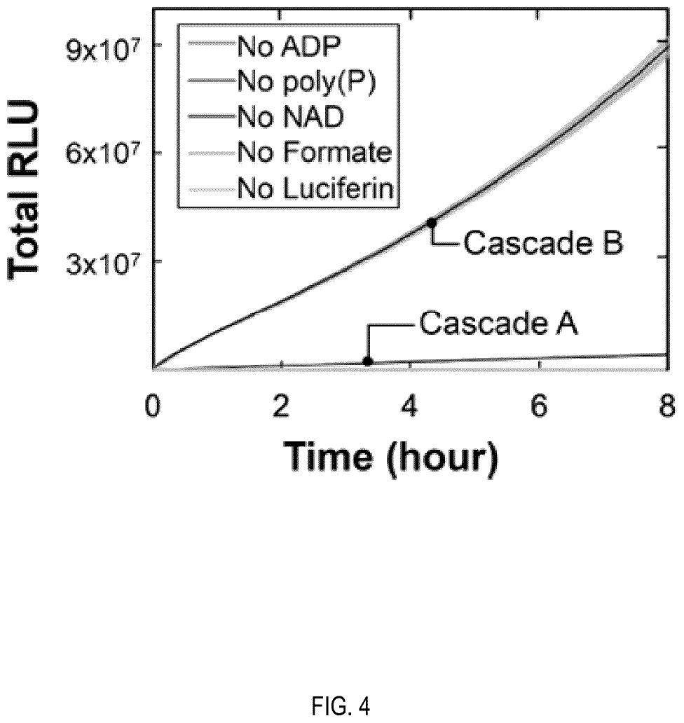

Referring now to FIG. 4, in an exemplary embodiment, a NADH-dependent dehydrogenase, PPNK, NADPH oxidases, and NADK were combined with luciferase and D-luciferin and concentrations of formate, poly(P), NAD⁺, ADP, and then monitored for the generation of an ATP product. Two exemplary cascades were prepared, as detailed in Table DI below:

TABLE II:
Enzyme and substrate concentration conditions for exemplary
synthetic enzymatic cascades consistent with embodiments of the
present disclosure.

| | “Cascade A” (mM) | “Cascade BA” (mM) |
|---|---|---|
| FDH | 0.001 | 0.00005 |
| PPNK | 0.001 | 0.001 |
| NADK | 0.001 | 0.003 |
| TPNOX | 0.001 | 0.00001 |
| Formate | 0.001 | 10 |
| NAD | 1 | 5 |
| MgCl₂ | 10 | 10 |
| Poly(P) | 10 | 10 |
| ADP | 1.25 | 3 |
| Luciferin | 10 | 10 |

Luminescence, indicating continuous ATP generation, was observed for over 8 h. As a control, removal of any one of the 5 substrates (formate, poly(P), NAD⁺, ADP, or luciferin) resulted in no luminescence, as seen at the bottom of FIG. 4. Without wishing to be bound by theory, these exemplary synthetic enzymatic cascade were formate-dependent and not driven by poly(P).

Exemplary embodiment “Cascade B” was constructed by reducing the concentrations of the enzymes responsible for fuel oxidation, FDH and TPNOX, due to their high turnover rates and low K_{M}^{NAD} and K_{M}^{NADPH} values. The concentration of NADK was increased as it had the lowest turnover rate of the enzymes in this exemplary enzymatic cascade. By adjusting the enzyme concentrations, the exemplary cascade “Cascade B” was able to generate a 5-fold higher total RLU rate with 5.6×10⁵ total RLU/h for “Cascade A” and 1.2×10⁷ total RLU/h for “Cascade B” in 200 μL volumes.

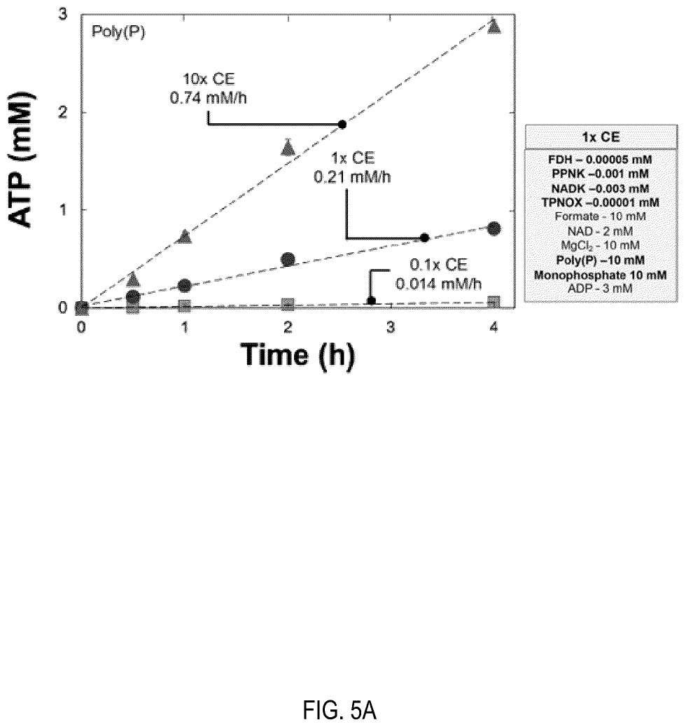

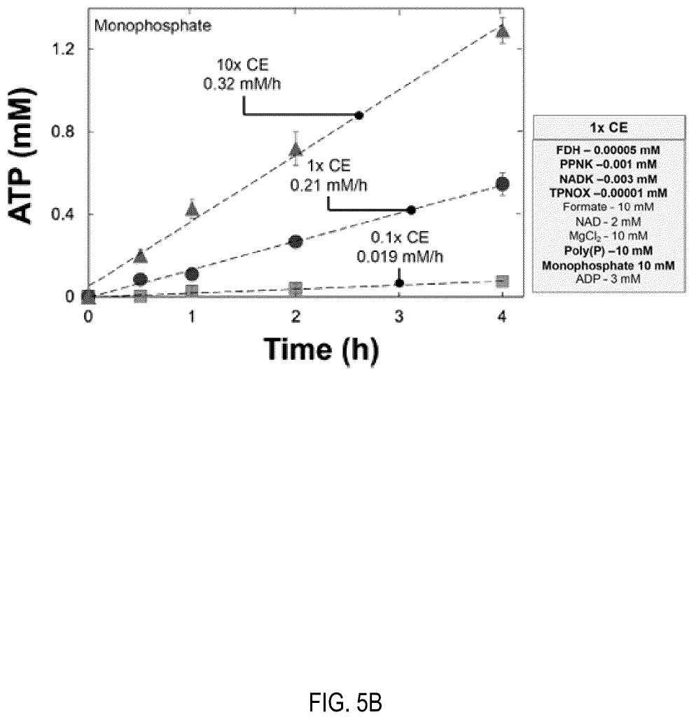

Relative luminescence in FIG. 4 was measured continuously, which complicates the quantification of the ATP production rate. Endpoint analyses with luciferase are more accurate for the determination of the rate of ATP regeneration. Referring now to FIGS. 5A-5B, ATP regeneration rates were determined through endpoint analyses for exemplary synthetic enzymatic cascade “Cascade B” (utilizing polyphosphates in FIG. 5A and monophosphates in FIG. 5B). The cascade enzyme concentrations (CE) from “Cascade B” were used for the 1× conditions. ATP production rates were also determined at 10 times the CE concentrations (10× CE), as well as a tenth of the concentrations (0.1× CE). Referring specifically to FIG. 5A, the polyphosphate cascade ATP production rate increased approximately 10-fold between 0.1× CE and 1× CE. Furthermore, the 10× CE had the highest ATP production rate, with 2.7 mM of ATP formed from an initial 3 mM ADP starting concentration or a 96% yield of ATP from ADP in 4 h (0.74 mmol/L/h in 200 μL volume). Referring specifically to FIG. 5B, similar patterns were seen with the monophosphate cascade, where increasing the CE concentrations led to higher overall ATP production.

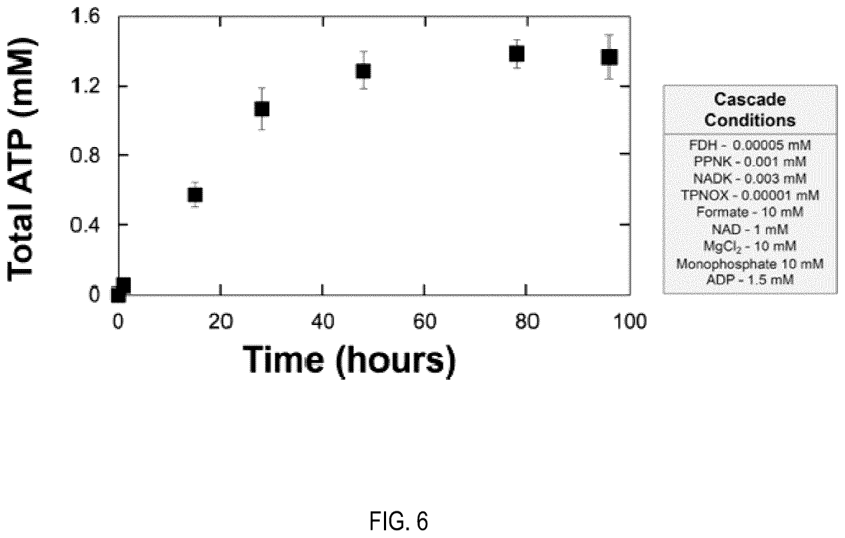

Referring now to FIG. 6, the limits of the exemplary 1× CE monophosphate cascade conversion were explored. The cascade was monitored for about 100 hours, with over 1.3 mM of ATP produced from 1.5 mM of ADP after 48 h (92% yield of ATP from ADP). These results demonstrated that formate oxidation by the synthetic enzymatic cascades of the present disclosure can be used to drive essentially complete conversion of ADP to ATP using monophosphate.

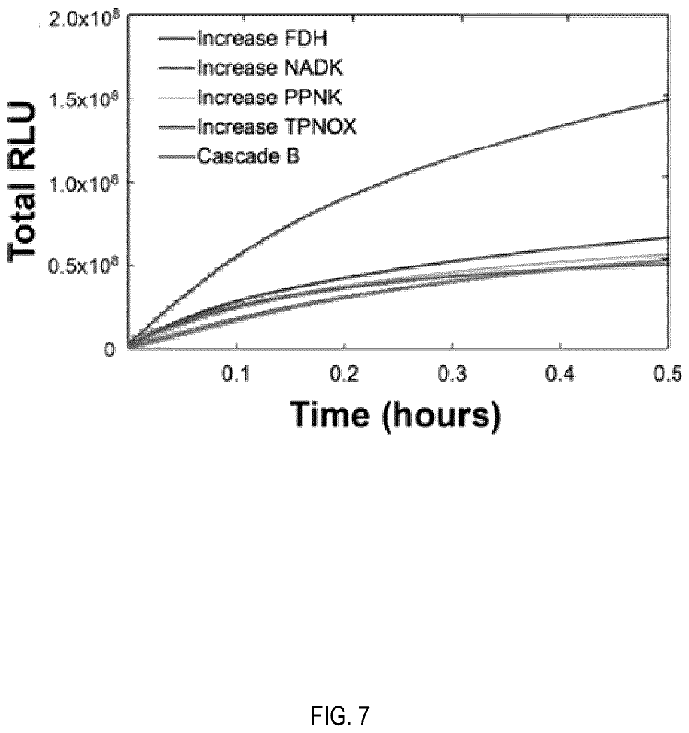

Referring now to FIG. 7, preliminary sensitivity analysis was performed in an effort to identify the extent to which certain enzymes in the synthetic enzymatic cascades consistent with embodiments of the present disclosure, e.g., “Cascade B,” were limiting on the generation of ATP products. Concentrations of each of the enzymes were doubled, and the effect on ATP formation was monitored. Increasing the concentration of TPNOX, PPNK, and NADK did not increase the ATP production rate. However, when FDH concentration was doubled, the cascade resulted in a 3-fold higher ATP regeneration rate (2.0×10⁸ total RLU/h in 200 μL). Without wishing to be bound by theory, in exemplary “Cascade B,” the activity of the FDH enzyme exhibits metabolic control over the rate of the cascade.

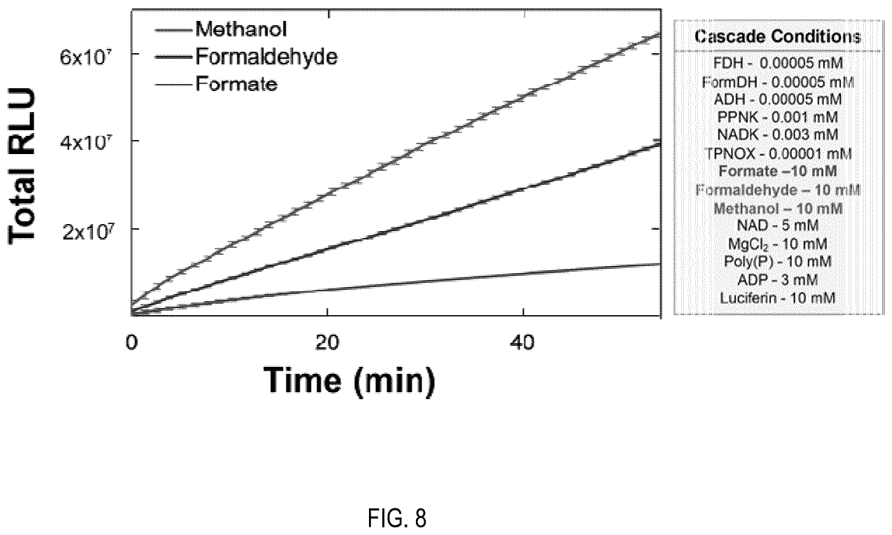

Referring now to FIG. 8, one benefit of the synthetic enzymatic cascades according to embodiments of the present disclosure is that a wide range of oxidoreductases could be used. In an exemplary embodiment, a multistep enzymatic fuel oxidation cascade including three dehydrogenase enzymes was prepared such that 3 moles of NADH were produced from every mole of a methanol fuel source, which was oxidized to CO₂. In this exemplary embodiment, an alcohol dehydrogenase oxidized methanol to formaldehyde, which was oxidized by formaldehyde dehydrogenase to formate, which in turn was oxidized to CO₂ by FDH. The additions of alcohol dehydrogenase and formaldehyde dehydrogenase enabled the testing of additional fuel sources for increasing the ATP production rate. Other “Cascade B” conditions, including polyphosphate, were chosen to enable comparisons with other exemplary embodiments discussed above. The highest ATP production rate was identified when 1 mM of methanol is added: 7.2×10⁷ total RLU/h in 200 μL. When formaldehyde was added, the ATP production rate was 4.4×10⁷ total RLU/h in 200 μL, and formate was 1.3×10⁷ total RLU/h in 200 μL under the conditions tested.

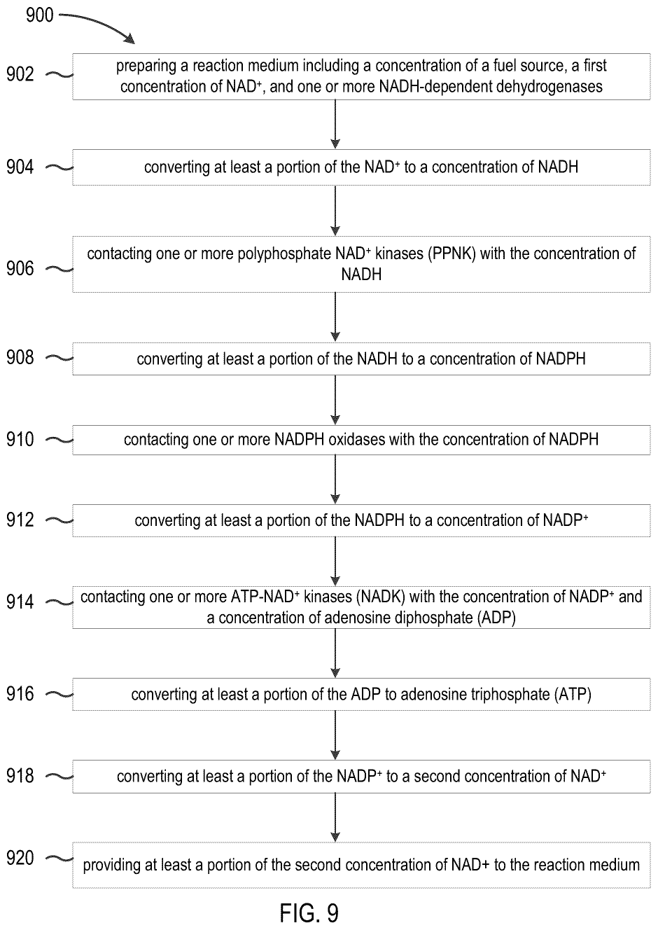

Referring now to FIG. 9, some embodiments of the present disclosure are directed to a method 900 of generating products via a synthetic enzymatic cascade. At 902, a reaction medium is prepared. In some embodiments, the reaction medium includes a concentration of a fuel source, a first concentration of NAD⁺, and one or more NADH-dependent dehydrogenases. As discussed above, in some embodiments, the fuel source includes formate, formaldehyde, methanol, glucose, glycerol, or combinations thereof. As discussed above, in some embodiments, the molar ratio of NADH-dependent dehydrogenase to fuel source is less than about 0.0001:1. In some embodiments, the molar ratio of NADH-dependent dehydrogenase to fuel source is between about 0.0001:1 and about 0.000005:1. In some embodiments, the molar ratio of NADH-dependent dehydrogenase to fuel source is between about 0.0001:1 and about 0.00001:1. In some embodiments, the concentration of the fuel source is maintained above about 0.1 mM. In some embodiments, the concentration of the fuel source is maintained above about 1 mM. In some embodiments, the concentration of the fuel source is maintained above about 10 mM. At 904, at least a portion of the concentration of NAD⁺ is converted to a concentration of NADH.

At 906, one or more PPNKs are contacted with the concentration of NADH. In some embodiments, the one or more PPNKs are from B. subtilis. At 908, at least a portion of the NADH is converted to a concentration of NADPH.

At 910, one or more NADPH oxidases are contacted with the concentration of NADPH. the NADPH oxidase includes a water-forming NADPH oxidase (TPNOX) from L. brevis, includes glucose-6-phosphate dehydrogenase (G6PDH), or combinations thereof. At 912, at least a portion of the NADPH is converted to a concentration of NADP⁺.

At 914, one or more NADKs are contacted with the concentration of NADP⁺ and a concentration of ADP. In some embodiments, the NADK is pigeon NADK, duck NADK, cat NADK, or combinations thereof. At 916, at least a portion of the ADP is converted to ATP. At 918, at least a portion of the NADP⁺ is converted to a second concentration of NAD⁺. At 920, at least a portion of the second concentration of NAD⁺ is provided to the reaction medium from step 902.

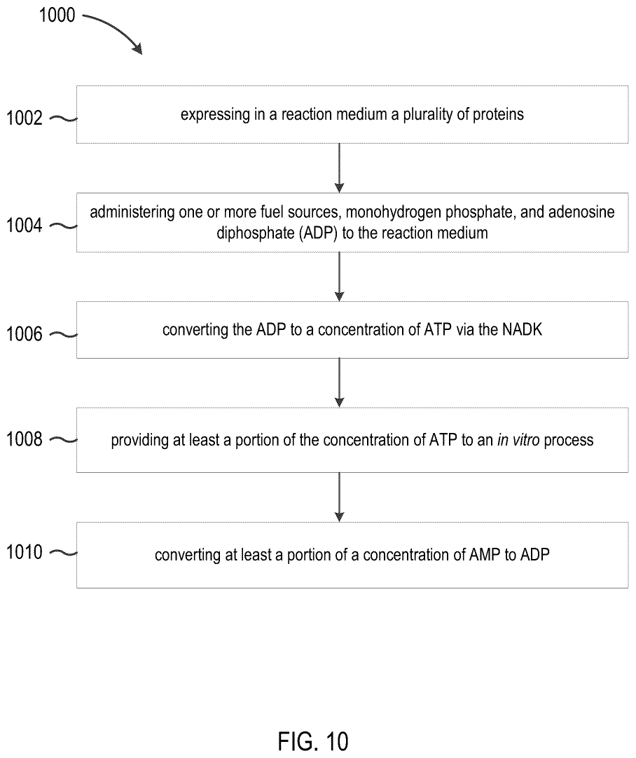

Referring now to FIG. 10, some embodiments of the present disclosure are directed to a method 1000 for continuously producing ATP. At 1002, a plurality of proteins are expressed in a reaction medium. In some embodiments, as discussed above, the plurality of proteins includes a formate dehydrogenase, a formaldehyde dehydrogenase, an alcohol dehydrogenase, pigeon NADK, a PPNK from B. subtilis, and TPNOX. At 1004, one or more fuel sources, monohydrogen phosphate, and ADP are administered to the reaction medium. In some embodiments, the one or more fuel sources includes methanol. In some embodiments, the molar ratio of NADH-dependent dehydrogenase to fuel source is less than about 0.0001:1. In some embodiments, the molar ratio of NADH-dependent dehydrogenase to fuel source is between about 0.0001:1 and about 0.000005:1. In some embodiments, the molar ratio of NADH-dependent dehydrogenase to fuel source is between about 0.0001:1 and about 0.00001:1. In some embodiments, the concentration of the fuel source is maintained above about 0.1 mM. In some embodiments, the concentration of the fuel source is maintained above about 1 mM. In some embodiments, the concentration of the fuel source is maintained above about 10 mM.

At 1006, the ADP is converted to a concentration of ATP via the NADK. At 1008, at least a portion of the concentration of ATP is provided to an in vitro process. In some embodiments, the in vitro process includes cell-free protein production, biomimetic artificial organelles, artificial cells, smart dust, or combinations thereof. In some embodiments, at 1010, the medium further comprises an adenosine monophosphate (AMP) phosphotransferase, and at least a portion of a concentration of AMP is converted to ADP.

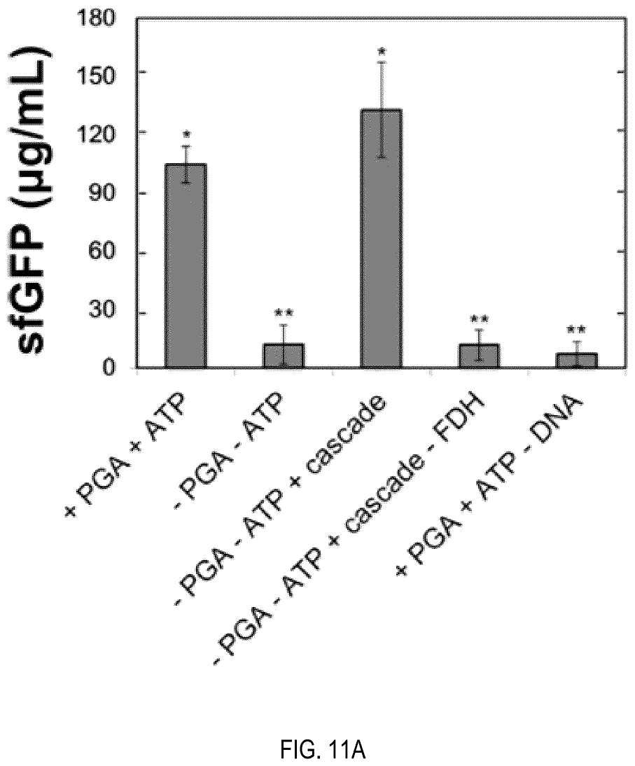

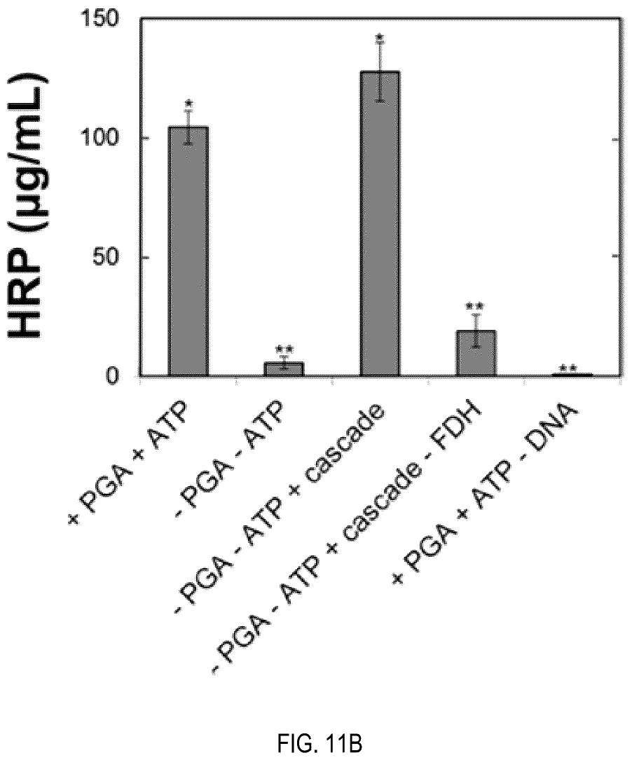

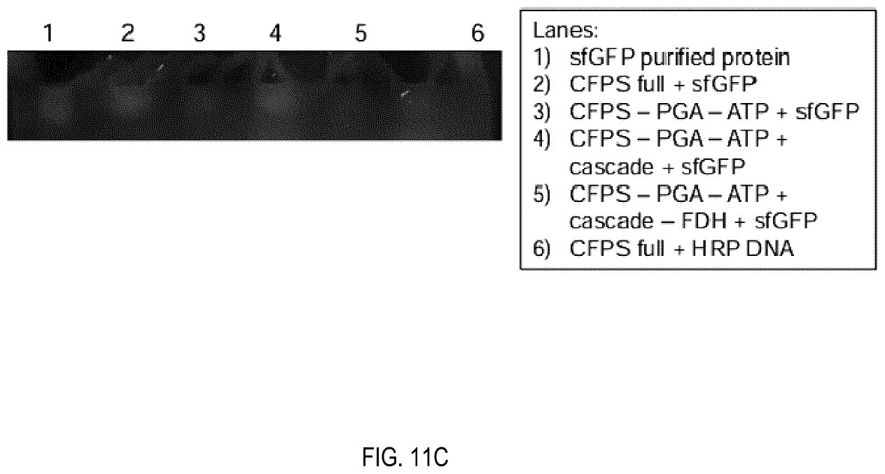

Referring now to FIGS. 11A-11C, exemplary synthetic enzymatic cascade “Cascade B” was implemented into a cell free protein synthesis system (CFPS). The CFPS system was used for the production of both superfolder green fluorescent protein (sfGFP) and horseradish peroxidase (HRP), and the concentrations of the proteins produced from CFPS were measured. When the normal CFPS phosphate donors, 3-phosphate glycerate (PGA) and ATP, were removed, there was no measurable recombinant protein expression. When the exemplary synthetic enzymatic cascade was implemented into the system (without PGA and ATP), there was higher protein expression for both sfGFP and HRP. This expression was lost when formate dehydrogenase was removed or when sfGFP and HRP DNA were removed. A native tris-glycine protein gel in FIG. 11C shows that functional fluorescent protein expressed only when the full NAD(P)(H) cascade was able to support the CFPS reactions.

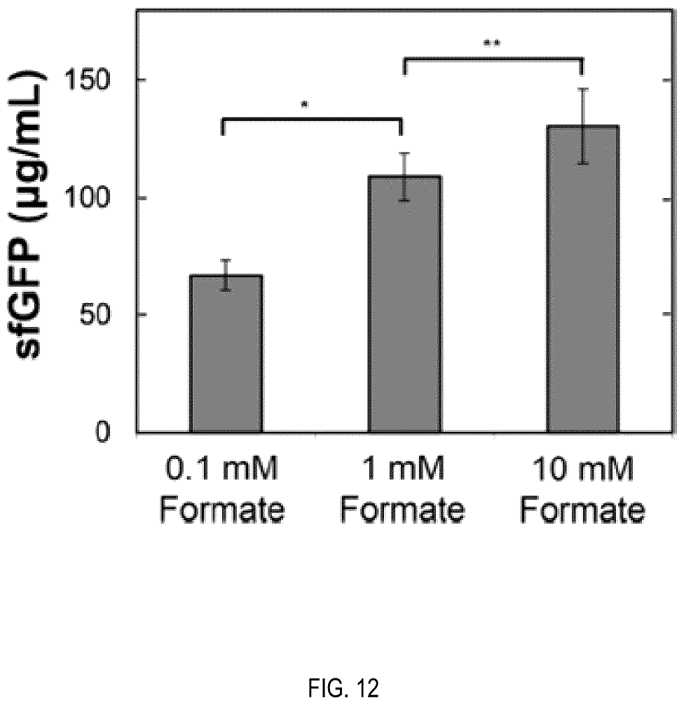

Referring now to FIG. 12, further analysis was performed to identify whether formate is a limiting reagent in a CFPS system. Varying formate concentrations were added in the synthetic enzymatic cascades consistent with embodiments of the present disclosure, e.g., “Cascade B,” and the CPFS was monitored for sfGFP. The sfGFP expression increased with increasing formate concentration, confirming that formate oxidation can control the rate of ATP regeneration for “Cascade B.”

### Materials

For overexpression, the enzymes were transformed into BL21(DE3) E. coli cells (New England Biolabs). Overnight cultures of BL21(DE3) E. coli cells harboring the DNA in pET20b plasmids and pET28a plasmid for FDH and sfGFP, respectively, were transferred into 50 mL of LB medium with 100 μg/mL ampicillin at 37° C. Overexpression of the enzymes was induced by adding isopropyl-β-thiogalactopyranoside (IPTG) to a final concentration of 1 mM at an optical density (OD) of 0.6 at 600 nm. After 3 h of induction, the cells were harvested and sonicated in wash buffer A for TPNOX (10 mM imidazole, 300 mM NaCl, and 20 mM Tris-HCl pH 7.4) and wash buffer B for all other enzymes (40 mM imidazole, 300 mM NaCl, and 20 mM Tris-HCl pH 7.4). The cells were centrifuged at 7000 rpm for 1 h and 4° C. The protein was purified using a nickel-nitrilotriacetic acid (Ni-NTA) column with continuous elution up to 500 mM imidazole.

PPNK activity was assayed by measuring the decreased absorbance of the reduction of NADPH to NADP⁺ at 340 nm by TPNOX. The reaction was carried out at 26° C. in a solution containing 10 mM Tris-HCl, pH 7.8, 10 mM MgCl₂, and 0.02 μg/μL TPNOX. Kinetic measurements of PPNK were conducted for substrates poly(P) and NADH with 0.04, 0.1, 0.3, 0.5, 0.8, and 1.2 mM NADH and 0.025, 0.05, 0.1, 0.25, 0.5, and 0.75 mM poly(P). Kinetic measurements of PPNK were conducted for substrates ATP and NADH with 0.2, 0.4, 0.8, 1.2, 1.5, and 2 mM NADH and 0.1, 0.25, 0.5, 1, 1.5, and 2 mM ATP. The reaction began when PPNK was added. The increasing product concentration was observed to be linear over the first 5 min, and each slope was performed in triplicate. A molar absorption coefficient of 6.22×10⁴ mol⁻¹ cm⁻¹ was used to convert absorption to molar concentration of NADPH.

ATP production was measured using firefly luciferase. The reaction began when NADK was added. The luminescence was measured in a plate reader in 26° C. in a solution containing 10 mM Tris-HCl, pH 7.8, and 10 mM MgCl₂. The ATP produced was calculated from luminescence using a calibration plot. In exemplary synthetic enzymatic cascade “Cascade A,” the CE concentrations were 0.001 mM each and luciferase concentration was 0.01 mM. The reagent concentrations were 10 mM formate, 1 mM NAD⁺, 10 mM MgCl₂, 10 mM poly(P), 1.25 mM ADP, and 10 mM luciferin. For exemplary synthetic enzymatic cascade “Cascade B,” the CE concentrations were: 0.00005 mM FDH, 0.001 mM PPNK, 0.003 mM NADK, and 0.00001 mM TPNOX. The luciferase concentration was 0.01 mM. The reagent concentrations were 10 mM formate, 5 mM NAD⁺, 10 mM MgCl₂, 10 mM poly(P), 3 mM ADP, and 10 mM luciferin. “Cascade A” and “Cascade B” were tested in 200 μL reaction volumes.

Exemplary cascades were tested with ADH and formDH. The enzyme concentrations were 0.001 mM PPNK, 0.003 mM NADK, 0.00001 mM TPNOX, 0.00005 mM FDH, 0.00005 mM, formDH, 0.00005 mM ADH, and 0.01 mM luciferase. The reagent concentrations were 1 mM NAD⁺, 10 mM MgCl₂, 5 mM poly(P), 1.25 mM ADP, and 10 mM luciferin. 1 mM formate, formaldehyde, and methanol were added separately to each corresponding reaction. The cascade was tested in 200 μL reaction volumes.

The procedure to express the S30-T7 lysate and the concentration of the components in the CFPS system. The final concentrations in the full CFPS system were 14 mM magnesium acetate, 50 mM potassium acetate, 155 mM ammonium acetate, 3% (v/v) polyethylene glycol, 40 mM 3-phosphoglycerate, 2.5 mM amino acids, 1.2 mM ATP, 1 mM GTP, 0.8 mM UTP, 30% (v/v) S30-T7 lysate, and 10 μg/mL mM HEPES-KOH (pH=8) to a final volume of 75 μL. Each CFPS sample was shaken at a temperature of 37° C. for 24 h. The sfGFP expression was monitored in the plate reader at an excitation of 485 nm and an emission of 510 nm, and the sfGFP concentration was determined from a standard curve. HRP expression was monitored in a solution with 100 μL of CFPS sample and 15 μL of ABTS and H₂O₂ such that the final concentration was 0.5 mM ABTS and 2.9 mM H₂O₂. The ABTS, H₂O₂, and CFPS sample was added to a 96-well plate, and the absorbance was measured at 405 nm. HRP expression was determined by monitoring the absorbance at 405 nm from a standard curve in 200 μL of 0.01 Tris-HCl buffer at pH 7.8 with 0.5 mM ABTS and 2.9 mM H₂O₂. The HRP concentrations tested were 0.1, 0.2, 0.4, 0.8, and 1 μg.

A tris-glycine protein gel was used to develop the native gel for the CFPS system with sfGFP. A 25 mM Tris, 250 mM glycine buffer was used. 10 μL of the CFPS sample, 10 μL of DI water, 5 μL of 4× LDS sample buffer (NUPAGE), and 2% (v/v) DDM was combined and centrifuged at 13000 rpm for 1 minute. 10 μL of the supernatant was loaded into each well and the gel was run on ice for 90 minutes at 100V. The protein gel was placed on a UV box to visualize the sfGFP.

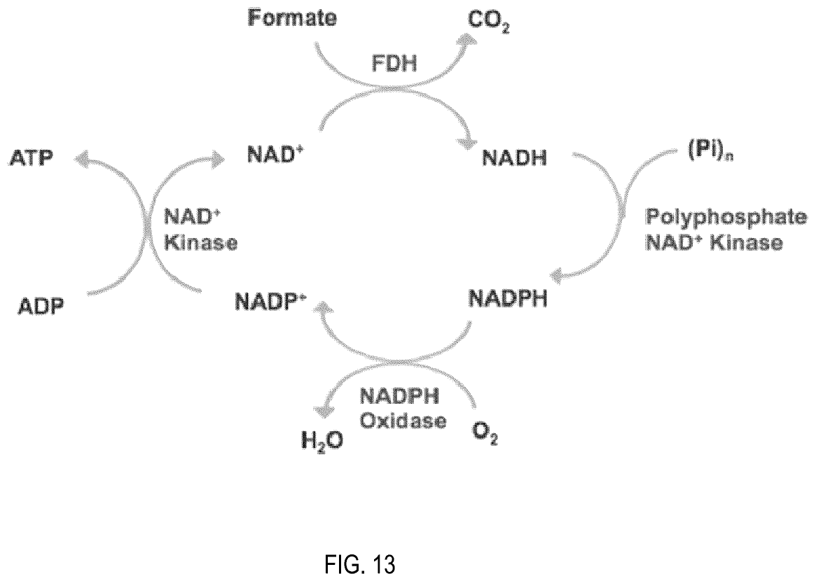

Methods and systems of the present disclosure are advantageous to provide a platform for continuously regenerating ATP for a variety of biotechnology applications. The methods and systems include an ATP regeneration enzyme cascade, e.g., as shown in FIG. 13, including an NADK that converts ADP and NADP+ to ATP and NAD+.

Most isoforms of the NADK demonstrate activity in the forward direction, i.e., with ATP as a substrate for the conversion of NAD+ to NADP. However, certain non-human NADKs, e.g., the pigeon, duck, and cat NADK, have a reasonable equilibrium constant and show reversible qualities.

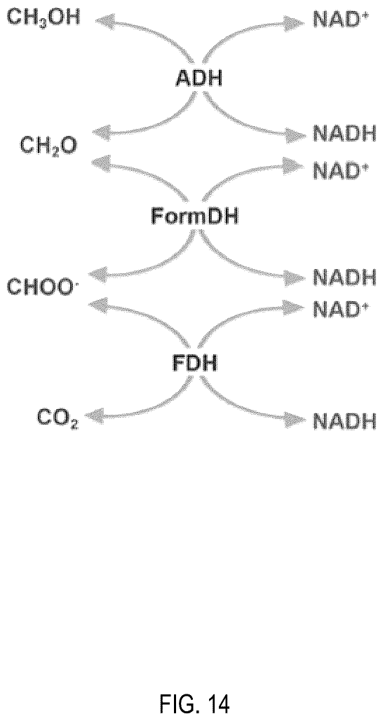

The enzyme cascade also includes formate dehydrogenases, e.g., from C. boidinii, which selectively oxidize formate fuels sources to CO₂ while producing NADH. One advantage to this cascade is other fuel sources can replace formate and still produce NADH. For example, it has been shown that a synthetic methanol oxidation cascade (see FIG. 14) can completely and selectively consume methanol and reduce NAD⁺ to NADH.

The enzyme cascade then includes a PPNK, e.g., from B. subtilis, which can selectively convert the NADH formed by the dehydrogenases to NADPH with a mono- and/or polyphosphate source. Finally, the enzyme cascade includes NADPH oxidase, e.g., a water-forming example from L. brevis, such that NADPH is converted back to NADP⁺. This enzyme is highly specific and can be the thermodynamic driving force of the cascade. One of the main challenges in assembling this cascade is avoiding cross-reactions. To address this, the enzymes used in the exemplary embodiments of the present disclosure are selective to their substrates.

As discussed above, state-of-the-art ATP regeneration methods generally use a constant supply of expensive phosphate donors such as creatine phosphate (for creatine kinase) or phosphoenol pyruvate (for pyruvate kinase). Other methods have exploited ATP synthase enzymes to generate ATP and further use a membrane to produce a proton gradient. In some embodiments, the embodiments of the present disclosure provide a low cost solution for ATP regeneration, e.g., for use in cell-free protein production, biomimetic artificial organelles, powering artificial cells, versatile ATP production from various fuel sources, synthetic pseudo-cell development for actuation and mobility, research tools for expanding genetic code, research tools for assembling viruses, tools for production of toxic and complex proteins, tools for high-throughput protein production, or combinations thereof.

However, the ATP regeneration cascades of the present disclosure do not use a membrane and rely on NAD(P)(H)-dependent reactions, which are cofactors in many biological reactions. Thus, in some embodiments, this cascade provides a technology for in vitro ATP regeneration driven by the oxidation of available, inexpensive fuels. This cascade continuously produced ATP for several hours. Embodiments of the present disclosure were able to reach an ATP production rate of 0.74 mmol/L/h for hours.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A method for continuously producing adenosine triphosphate (ATP), comprising:
expressing one or more NADH-dependent dehydrogenases, one or more polyphosphate NAD⁺ kinases (PPNK), one or more NADPH oxidases, and one or more ATP-NAD⁺ kinases (NADK);
oxidizing one or more fuel sources while converting NAD⁺ to NADH via the NADH-dependent dehydrogenases;
converting NADH to NADPH via the PPNKs;
converting NADPH to NADP⁺ via the NADPH oxidases; and
converting NADP⁺ to NAD⁺ while converting one of adenosine diphosphate (ADP) or adenosine monophosphate (AMP) to ATP via the NADKs.

2. The method according to claim 1, wherein the NADH-dependent dehydrogenase includes formate dehydrogenase, formaldehyde dehydrogenase, alcohol dehydrogenase, or combinations thereof.

3. The method according to claim 2, wherein the molar ratio of NADH-dependent dehydrogenase to fuel source is less than about 0.0001:1.

4. The method according to claim 2, wherein the NADH-dependent dehydrogenase includes formate dehydrogenase, formaldehyde dehydrogenase, and alcohol dehydrogenase.

5. The method according to claim 2, wherein the NADH-dependent dehydrogenase includes formate dehydrogenase from C. boidinii.

6. The method according to claim 1, wherein the one or more PPNKs are from B. subtilis.

7. The method according to claim 1, wherein converting NADH to NADPH via the PPNKs occurs in the presence of a monophosphate compound.

8. The method according to claim 1, wherein the NADPH oxidase is a water-forming NADPH oxidase (TPNOX) from L. brevis, includes glucose-6-phosphate dehydrogenase (G6PDH), or combinations thereof.

9. The method according to claim 1, wherein the NADK is pigeon NADK, duck NADK, cat NADK, or combinations thereof.

10. The method according to claim 1, wherein the fuel source includes formate, formaldehyde, methanol, glucose, glycerol, or combinations thereof.

11. The method according to claim 10, wherein the concentration of the fuel source is maintained above about 10 mM.

12. A method of generating products via a synthetic enzymatic cascade, comprising:
preparing a reaction medium including a concentration of a fuel source, a first concentration of NAD⁺, and one or more NADH-dependent dehydrogenases;
converting at least a portion of the NAD⁺ to a concentration of NADH;
contacting one or more polyphosphate NAD⁺ kinases (PPNK) with the concentration of NADH;
converting at least a portion of the NADH to a concentration of NADPH;
contacting one or more NADPH oxidases with the concentration of NADPH;
converting at least a portion of the NADPH to a concentration of NADP⁺;
contacting one or more ATP-NAD⁺ kinases (NADK) with the concentration of NADP⁺ and a concentration of adenosine diphosphate (ADP); and
converting at least a portion of the ADP to adenosine triphosphate (ATP).

13. The method according to claim 12, further comprising:
converting at least a portion of the NADP⁺ to a second concentration of NAD⁺; and
providing at least a portion of the second concentration of NAD⁺ to the reaction medium.

14. The method according to claim 12, wherein the fuel source includes formate, formaldehyde, methanol, glucose, glycerol, or combinations thereof.

15. The method according to claim 14, wherein the molar ratio of NADH-dependent dehydrogenase to fuel source is less than about 0.0001:1.

16. The method according to claim 12, wherein the NADK is pigeon NADK, duck NADK, cat NADK, or combinations thereof.

17. A method for continuously producing adenosine triphosphate (ATP), comprising:
expressing in a reaction medium a plurality of proteins including:
a formate dehydrogenase, a formaldehyde dehydrogenase, an alcohol dehydrogenase, pigeon ATP-NAD⁺ kinase (NADK), a polyphosphate NAD⁺ kinase (PPNK) from B. subtilis, and triphosphopyridine nucleotide oxidase (TPNOX);
administering one or more fuel sources, monohydrogen phosphate, and adenosine diphosphate (ADP) to the reaction medium;
converting the ADP to a concentration of ATP via the NADK; and
providing at least a portion of the concentration of ATP to an in vitro process,
wherein the one or more fuel sources includes methanol.

18. The method according to claim 17, wherein the in vitro process includes cell-free protein production, biomimetic artificial organelles, artificial cells, smart dust, or combinations thereof.

19. The method according to claim 17, wherein the medium further comprises an adenosine monophosphate (AMP) phosphotransferase, and the method further comprises:
converting at least a portion of a concentration of AMP to ADP.

20. The method according to claim 19, wherein:
the molar ratio of NADH-dependent dehydrogenase to fuel source is less than about 0.0001:1, and
the concentration of the fuel source is maintained above about 10 mM.
